(19) United States
(12) Patent Application Publication
(10) Pub. No.: US 2015/0057513 A1
(43) Pub. Date: Feb. 26, 2015

(54) Minimally Invasive Stress Sensors and Methods

(71) Applicant: ARIZONA BOARD OF REGENTS, A BODY CORPORATE OF THE STATE OF ARIZONA ACTING FOR AND ON BEHALF OF AR, Scottsdale, AZ (US)

(72) Inventors: Jeffrey LaBelle, Tempe, AZ (US); Katherine Ruh, Tempe, AZ (US); Brittney Haselwood, Chandler, AZ (US)

(73) Assignee: Arizona Board of Regents on Behalf of Arizona State University, Scottsdale, AZ (US)

(21) Appl. No.: 14/378,454

(22) PCT Filed: Mar. 8, 2013
(86) PCT No.: PCT/US13/30007

§ 371 Date: Aug. 13, 2014

Related U.S. Application Data
(60) Provisional application No. 61/646,812, filed on May 14, 2012.

(51) Int. Cl. A61B 5/16; A61B 5/00; A61B 5/1473

(52) U.S. Cl. CPC A61B 5/165; A61B 5/1473; A61B 5/685
USPC 600/345

(57) ABSTRACT

Methods and devices to continuously measure electrochemical activity of one or more biochemical or molecular markers (FIG. 9). A substrate having electronics for measuring electrochemical activity and a plurality of electrodes such that the electrodes are in contact with the subcutaneous layer are attached to subject's skin or intravenously. The devices measure a biochemical process associated with one or more biochemical or molecular markers in vivo by detecting an electrochemical signal in subcutaneous layer (or intravenously) using the plurality of electrodes.

### CROSS-REFERENCE TO RELATED APPLICATIONS

This application claims priority to U.S. Provisional Patent Application Ser. No. 61/646,812 filed on May 14, 2012.

### BACKGROUND OF THE INVENTION

According to the CDC, approximately 1.7 million Traumatic Brain Injuries (TBIs) occur annually in the United States. There are two general types of TBI: closed (e.g. whiplash, blunt trauma; where the brain hits the inside of the skull) and penetrating (e.g. gun shots, stabbing; where the brain has been pierced by a foreign object) injuries. Of annual TBIs, 52,000 lead to deaths, 275,000 hospitalizations, and 1,365,000 emergency and urgent care visits. In 2000, medical costs associated with TBIs were estimated to be $60 billion, while in 2010 costs rose to $76.5 billion, again for approximately 1.7 million patients. Unfortunately, there are many TBIs that go unreported due to the mild severity of the TBI; 75% of TBIs are of the mild variety. TBI can range from mild (minute headache with minimal to no other symptoms), to severe (loss of consciousness and serious brain damage).

Depending on the severity of the injury, mental and cognitive functions such as thinking, sensation, language, and emotion may be affected. Particularly if the patient sustained injury at an older age, TBI can predispose the patient to Alzheimer's and Parkinson's disease in addition to epilepsy; even if the patient were not already predisposed. The most dangerous kind of TBI is the one which goes untreated; extended TBIs (many small mild traumas) may accumulate into neurological and cognitive dysfunctions, while many more severe TBIs sustained in a short time may lead to life-altering damage or death [1].

Products which are directed to TBI monitoring include: Parc's flexible intracranial pressure sensor [2], varieties of military helmets which change color when pressure is sensed [3], or can measure shock [4], an extracorporeal protein ELISA sensor from the University of Florida [5], and Medtronic's Continuous Glucose Monitoring system [6].

An advantage of the Parc flexible pressure sensor is that the monitoring is occurring in the environment of injury. However, the placement of this sensor requires invasive surgery for a patient with pre-existing trauma (due to the initial head injury). The military helmets have likely been funded due to the Army's decision in 2006 to create a taskforce to oversee preventions and treatments of soldiers who sustain TBI in the Middle East [7]. These helmets can also be used in sporting activities and are not invasive, but these measure bulk forces and are not necessarily indicative of injury and do not monitor the injury itself.

The TBI nanosensor being developed at the University of Florida is a protein bound to a nanosphere. Unfortunately, the actual testing occurs in a handheld ELISA device requiring media like spinal fluid, which is somewhat invasive to obtain. This sensor may be very sensitive, but it most resembles a self-monitoring blood glucose (SMBG) device which is not continuous.

The last sensor from Medtronic is a subcutaneous continuous sensor. Advantages of this sensor include that it is connected to a drug delivery device (an insulin pump via wireless interaction); however, this is specifically for diabetes management.

### SUMMARY OF THE INVENTION

Embodiments herein relate to methods to continuously measure electrochemical activity of one or more biochemical or molecular markers associated with stress by attaching a substrate having electronics for measuring electrochemical activity and a plurality of electrodes, such that the electrodes are in contact with the subcutaneous layer of a subject's skin, and measuring a biochemical process associated with the one or more biochemical or molecular markers in vivo by detecting an electrochemical signal in the subcutaneous layer using the plurality of electrodes.

Embodiments also relate to devices adapted to measure electrochemical activity of a biochemical or molecular marker, the devices having a substrate with electronics adapted for measurement of electrochemical activity and a plurality of electrodes, the electrodes being attached to the substrate, operably connected to the electronics, and adapted to penetrate the skin to a subcutaneous layer.

Various other purposes and advantages of the invention will become clear from its description in the specification that follows. Therefore, to the accomplishment of the objectives described above, this invention includes the features hereinafter fully described in the detailed description of the preferred embodiments, and particularly pointed out in the claims. However, such description discloses only some of the various ways in which the invention may be practiced.

### BRIEF DESCRIPTION OF THE DRAWINGS

FIG. 1 depicts the synthesis of the catecholamines in the human body in a series of enzymatic reactions.

FIG. 2 depicts, on the left, a generic example of the voltage perturbation, V(t), and the system's response in the form of current as functions of time, I(t). The phase shift, φ, is determined by measuring the distance between the peaks of the V(t) and I(t) curves. On the right is a representation of a Nyquist plot which is a function of imaginary impedance (ZI) and real impedance (ZR). The phase shift in the Nyquist plot is the angle between the line created and the x-axis.

FIG. 3 illustrates a double layer capacitor (Cdl), which is created when the linker is attached to the surface of the hydrophilic electrode (obtained by thorough cleaning), the charge transfer resistance (RCT) is the current flow created when a redox reaction occurs in the system, the Warburg impedance (W) occurs due to diffusion of the redox species in the system, RSDL is the solution resistance at the double layer (characteristic of the fluid), and σ is a value related to the Warburg resistance.

FIG. 4 is a CV graph of 277.9 mM Dopamine in 100 mM ferri-, 100 mM ferrocyanide (Redox Probe) on Glassy Carbon Electrode.

FIG. 5 is a CV graph of Epinephrine, Norepinephrine, and Dopamine.

FIG. 6 is an AMP-it of DAHCl at 0.8V potential. Inlaid graph of Current (0.1 A) v. [DAHCl] (M) as different times during the AMP-it assay. This simulates a continuous times sensing assay.

FIG. 7 depicts enzymes immobilized on a gold disk surface for specific binding and sensing of substrate or antigen, i.e. target molecule.

FIG. 8 depicts a Glassy Carbon Electrode with a reference and counter electrode secured onto a cut pipette tip with a sample inside. This is the set up for running most electrochemical assays. Also depicted is the top view of only the Glassy Carbon surface.

FIG. 9 illustrates the base of the needle sensor embodiment. The base is a Print Circuit Board with the dark area being the copper.

FIG. 10 shows a needle and adhesive assembly device embodiment.

FIG. 11 shows the comparison between concentration at a sensitivity of 1.0E-03 and the current that was found for each needle size. This graph also shows a comparison between two concentration experiments of epinephrine vs. blood with each needle. The purified data is seen to have much more current then the blood data.

FIG. 12 shows the first concentration and how purified data and blood data for each needle size compare with each other. The current for the blood is much small and does not match up with the purified data. The current for the blood data has a negative slope form where as the purified does not.

FIG. 13 shows the second concentration and how the purified data and blood data for each needle size compare with each other. The current for the blood data is smaller then the current for the purified data. The data does not conform to the same layout. For example the 18 gauge needle shows a large difference between the purified and the blood data

FIG. 14 shows the third concentration and how the purified and blood data for each needle size compare with each other. The current for the blood data is smaller then the current for the purified data. The data for both show the same kind of form between the needles even though the currents are different.

FIG. 15 shows (inlaid) an Amp-it of DA with the voltage applied at the oxidation peak of the CV, 0.52V. The outer graph is a calibration curve which plots current versus concentration of DA at different times during the AMP-it: A (2 sec), B (12 sec), C (20 sec).

FIG. 16 is a calibration curve which correlated the impedance to the concentration of Dopamine in purified solution at 4590 Hz.

FIG. 17 is a calibration curve which correlated the impedance to the concentration of Dopamine in blood solution at 4590 Hz.

FIG. 18 is a calibration curve which correlated the impedance to the concentration of Epinephrine in purified solution at 3711 Hz.

FIG. 19 is a calibration curve which correlated the impedance to the concentration of Epinephrine in blood solution at 4590 Hz.

FIG. 20 is a calibration curve which correlated the impedance to the concentration of Norepinephrine in purified solution at 1465 Hz.

FIG. 21 is a calibration curve which correlated the impedance to the concentration of Norepinephrine in blood solution at 3711 Hz.

FIG. 22 depicts an intravenous sensor embodiment in which (A) depicts electrodes in a device (B) that is implantable in a blood vessel (C) of, for example, an arm (D).

FIG. 23 illustrates a protein recognition element with which the catecholamines can be specifically measured. The proteins to be used mimic the ones naturally found in the human body. Using immobilization chemistry and a very sensitive assay, electrochemical impedance spectroscopy, the catecholamines can be measured with a lower limit of detection in the femptomolar range.

FIG. 24 show that EIS has been implemented to characterize each catecholamine in purified and blood sample bench-top experiments. Optimal binding frequencies have also been determined to be used in future integration methods.

FIG. 25 shows the physiological levels of additional biomarkers relating to stress and trauma.

FIG. 26 depicts data from sensor material design factor testing in blood.

FIG. 27 shows data from electrochemical experiments that have been run to prove the feasibility of detecting Norepinephrine using the PNMT enzyme through the application of mesoporous carbons.

FIG. 28 is a graph showing that pressure was monitored over time to determine if the PEN material causes pressure changes as a 25% blood solution is passed through the material. It was determined that no significant pressure changes occurred over the time monitored.

FIG. 29 shows flow rate measurements.

FIG. 30 compares physiological levels of additional biomarkers relating to stress and trauma.

### DETAILED DESCRIPTION OF THE INVENTION

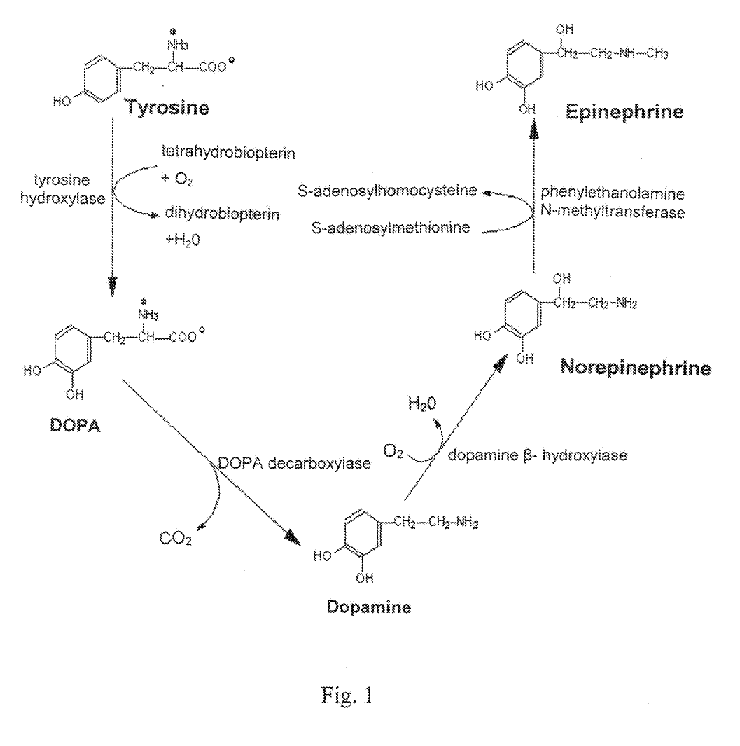

Epinephrine and Norepinephrine are neurocrines or catecholamines involved in catalyzing the fight or flight response in the human body, among other functions such as inflammation response. Both Epinephrine and Norepinephrine are produced in the Adrenal Medulla and bind to adrenergic receptors; Norepinephrine has a greater affinity for α-receptors and Epinephrine has a greater affinity for β₂-receptors. The catecholamines are synthesized in the human body in a cascade as seen in FIG. 1. First the side chains of Tyrosine, the amino acid, are modified by a sequence of enzymatic reactions to form Dopamine, Epinephrine, and Norepinephrine.

As depicted in FIG. 1, the enzyme which converts Norepinephrine into Epinephrine is phenylethanolamine N-methyltranferase (PNMT). This enzymatic reaction will not proceed without the prescence of a co-factor, S-(5′-Adenosyl)-L-methionine chloride (SAM). It is proposed that SAM is a “functional response element” which initiates the expression of the PNMT gene and allows the enzyme to become active. Due to the characteristics of the enzyme, PNMT will take Norepinephrine as its specific substrate and produce Epinephrine as shown in FIG. 1. The concentration of these catecholamines can be determined in multiple bodily fluids, however, the most reliable and reproducible data obtained has been in blood. Blood plasma levels of the catecholamines are very low in comparison to concentrations of other constituents in blood such as Oxygen or hemoglobin.

TABLE 1
The table below contains the blood plasma concentrations
of each of the main catecholamines in picograms/milliliter
(pg/mL) as found experimentally in published literature.
Physiological Levels of Catecholamines

| | Plasma Concentration |
|---|---|
| Catecholamine | (pg/mL) |
| Dopamine | 98 ± 20 |
| Epinephrine | 64 ± 5 |
| Norepinephrine | 203 ± 10 |

Table 1 contains the blood plasma levels of the catecholamines in pg/mL. If one wanted to detect the presence of the catecholamines such as Norepinephrine in the blood, one could feasibly create a specific and sensitive biosensor using the binding of Norepinephrine to the PNMT enzyme as the signal. As this signal is very small, a new electrochemical technique called Electrochemical Impedance Spectroscopy (EIS) is used to collect the catecholamine concentration in the blood data.

This new technique is required to determine blood plasma catecholamine concentrations due to the lack of sensitivity of other well-established assays in the field (such as Amperometric i-t curve technique commonly used in SMBGs). Other advantages of this technique is that it is label-free thereby making it a cheaper approach with an advantage over the traditional sandwich ELISA technique used by the University of Florida's device.

EIS is the analysis of electrical resistance in a system. This method of measurement is sensitive to the “surface phenomena and bulk properties.” For example, this method can deduce signals from changes to its surface such as something binding to it in some fashion (adsorption or immobilization of protein), or if a state change is occurring. What makes this method valuable is that it does not require labeling of the targets to be measured (e.g. dyes or radioactive labels). The EIS technique works by measuring the impedance, Z, of a system through a frequency sweep at a particular voltage. The instrument which executes this data collection applies a “voltage perturbation” close to the user defined voltage, usually related to the formal potential mentioned later, and the machine measures the current response of the system following this model:

$$Z = \frac{V ( t )}{I ( t )} = \frac{{V}_{0} sin ( 2 π f t )}{{I}_{0} sin ( 2 π f t + ϕ )} \qquad ( Eqn . 1 )$$

Where Z is the impedance calculated from the voltage applied as a function of time V (t) and current as a function of time I (t). The maximum current and voltage values are represented by I₀ and V₀, respectively while f represents frequency, t is time, and φ is the phase shift between the current and voltage signals.

TABLE 2

| Impedance | | Phase | Frequency |
|---|---|---|---|
| element | Definition | angle | dependence |
| R | Z = R | 0° | No |
| C | ${Z}_{C} = \frac{1}{jω C}$ | 90° | Yes |
| CPE | ${Z}_{CPE} = \frac{1}{{A ( j ω )}^{a}}$ | 0-90° | Yes |
| W (infinite)^{a} | ${Z}_{W} = \frac{σ}{\sqrt{ω}} ( 1 - j )$ | 45° | Yes |
| | $σ = \frac{R T}{{n}^{2} {F}^{2} \sqrt{2}} ( \frac{1}{\sqrt{{D}_{0}} {c}_{0}} + \frac{1}{\sqrt{{D}_{R}} {c}_{R}} )$ | | |

The above tabulates the affects of system elements, such as a capacitor, has on the phase shift, φ, as quantified in degrees. Also tabulated is the nature of the element's dependency on frequency. This means that the phase may be different at various frequencies.

The phase shift occurs when a capacitive or inductive element is present in the system thereby causing complex (real and imaginary) impedance. The data collected can therefore be represented in one of two ways: (1) in a Bode plot with the magnitude of the impedance and phase shift (φ) as functions of frequency or (2) a Nyquist plot which a graphical representation of the real vs. imaginary impedance where the phase shift is the angle between the line and the x-axis.

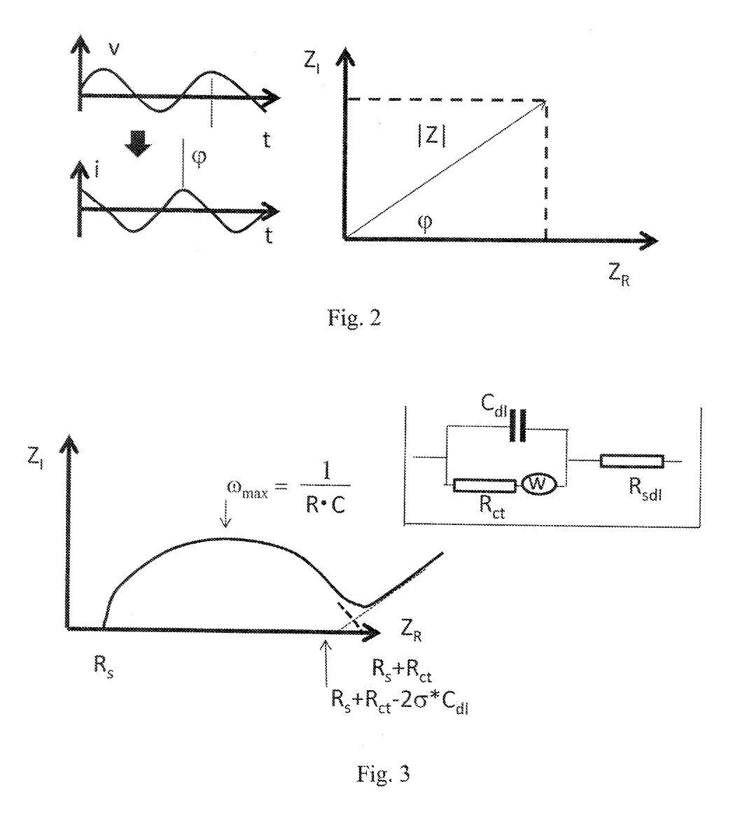

FIG. 2 illustrates the definition of phase shift and a general representation of the Nyquist Plot. As previously mentioned, the phase shift can be affected by a capacitive or inductive element within the system as quantified in Table 2. The second table also provides definitions of possible system elements. This is useful as some molecules act as resistors, while others act like capacitors, in the system. It is important to be able to quantify these system elements because it is simple to make an equivalent circuit for the system. For example, a common model is known as the Randles' circuit. This circuit is a simplification for the electrode-electrolyte configuration. This may occur when placing a linker (a 16 Carbon chain which binds to the electrode surface and acts as an anchor to which for protein can bind) on an electrode surface before immobilizing a protein.

The result of this system is a Nyquist shape known as the Warburg, whose equivalent circuit model is depicted in FIG. 3. The model accounts for a double layer capacitor (C_{dl}) which is created when the linker is attached to the surface of the hydrophilic electrode (obtained by thorough cleaning of the surface). After the linker is placed on the electrode surface, a protein such as an antibody or enzyme may be immobilized onto the electrode.

There are two main applications of EIS: (1) to determine the impedance of a sample to be later (with analysis) represented as a function of the concentration of the species/target versus time, and (2) to utilize the negative of the desired target as an immobilized recognition element placed onto the working electrode to in a substrate-enzyme, or antigen-antibody manner (i.e. detect protein-target binding interaction). The second approach is particularly useful for a biosensor application since impedance can be directly related to a concentration based on the circuit and concentration modeling.

Also, the time element in the second approach would be useful in making a continuous concentration-impedance sensor. However, before a sensor can be developed through the use of EIS, other preliminary and basic electrochemical assays must be performed on both the target (Norepinephrine) and sensing species (PNMT). The basic and widely used electrochemical assays used in publications today include Cyclic Voltammetry (CV), Amperometric i-t Curves (AMP i-t) and Square Wave Voltammetry (SQW) [13]. Cyclic voltammetry, also known as potentiometry, measurers a current between two electrodes as a voltage or potential is applied to the sample (as a sweep/cyclic function between two specified voltages).

The difference of current can be measured between the reference electrode and the working electrode while the counter electrode provides the signal, in this case voltage sweep. As the working electrode is typically a metal/conducting material that does not take part in the chemical reaction, this is known as an “inert-indicator-electrode” meaning that it is only the point of measurement in the system. A CV is a graph of current in amps versus voltage in volts. The peaks represent when the sample has lost its maximum amount of electrons (maximum oxidation state) or gained the maximum number of electrons (maximum reduction state). Also, the formal potential is where one could draw the center of mass of a CV curve; this is used in EIS and the voltage to be defined by the user as a parameter of the impedance experiment.

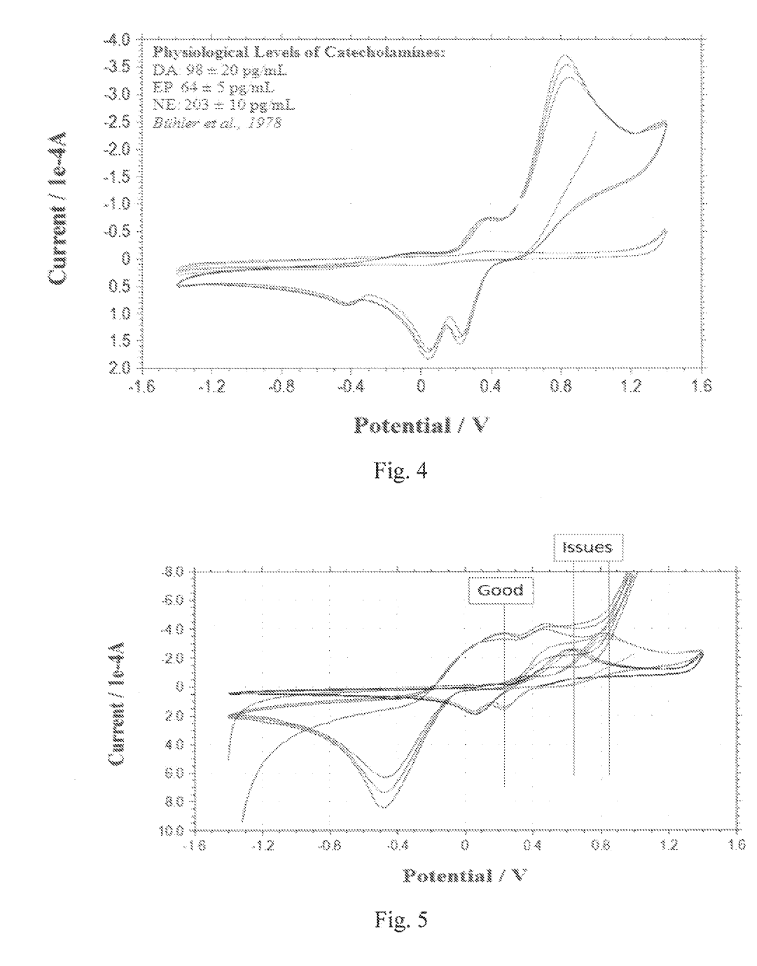

One oxidation peak and one reduction peak is a very simple case, many substances have very characteristic and multiple peaks, such as the catecholamines. FIG. 4 is an example of a characteristic CV curve for dopamine in Redox probe, the relatively flat curve around zero current being the blank, or simply the Redox Probe. As seen in FIG. 4, it is possible to have local maxima and minima with respect to oxidation and reduction which can be compared to characteristics of yet another substance as seen in FIG. 5.

If the desire were to create a sensor that performed electrochemical assays on human blood, there would be convolution of the signal received at certain voltages (indicated in FIG. 5 with vertical lines). As shown, many peaks for multiple substances overlap, thus an electrochemical test for a sensor to simultaneously detect these three catecholamines would have issues with being non-specific. This assay would not be used due to its lack of sensitivity based especially since the order of magnitude the catecholamines exist in the blood is pg/mL.

Amperometric i-t curves are more sensitive than CVs, as it measures current as a constant voltage is maintained. This constant voltage is the maximum oxidation or reduction peak that is characteristic of the substance being tested. In other words, the CV must first be run to determine the voltage which may be used in the AMP-it.

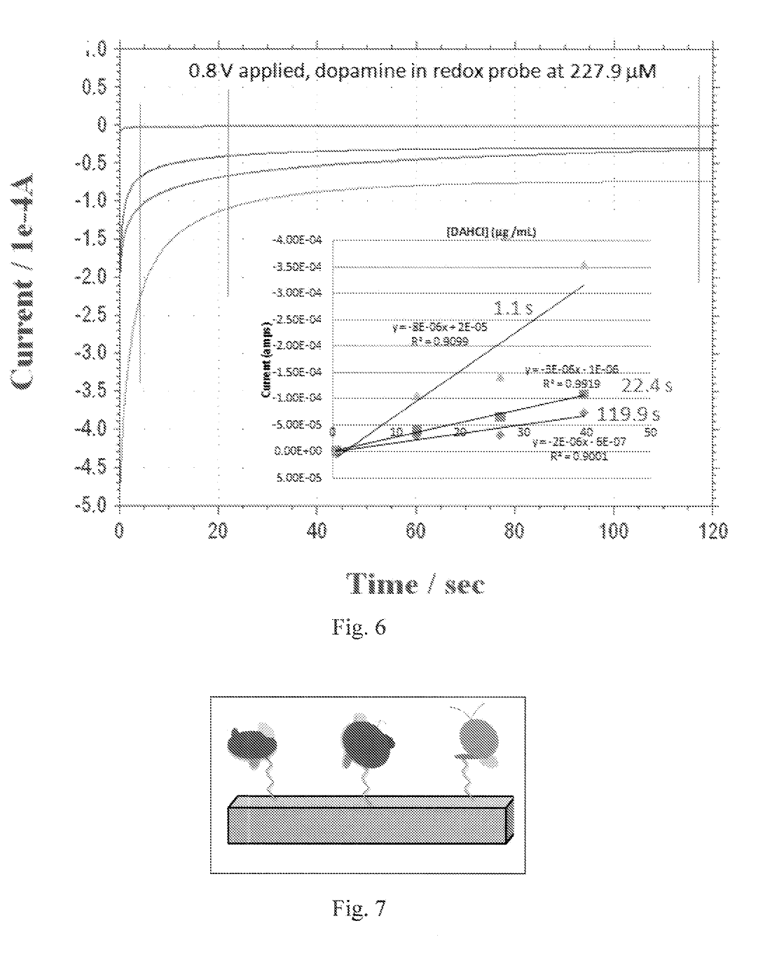

Amperometric i-t curves, also known as amperometry, measures the amount of current that flows between the working electrode and the reference electrode given the previously discussed constant voltage [13]. This electrochemical assay is useful for monitoring changes in current over time of a sample while a voltage is being applied [6]. FIG. 6 depicts an example of the output received from the AMP-it assay performed on a concentration gradient of Dopamine Hydrochloride. The inset table is generated from maximum change in the slope of the AMP i-t curve. This kind of electrochemical assay would be helpful if applied in a sensor that needed to read a particular level of a substance over time. This would be beneficial for something like a continuous glucose sensor if the electrochemical characteristics of glucose were programmed to the sensor. Then it stands to reason that it could monitor changes in the blood levels of the catecholamines over time if programmed correctly. However, the need for specificity is still not met; it is for this reason that EIS is the next step in creating a specific and sensitive continuous sensor.

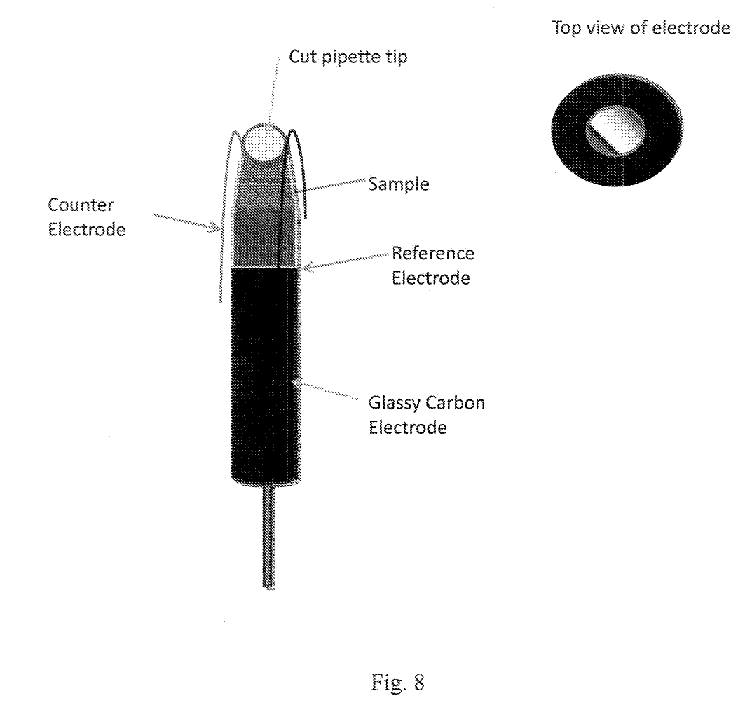

As seen in the data provided, the targets or substrates have been electrochemically identified via less sensitive, but more established techniques. To create a sensitive and specific continuous time sensor, the next steps will include immobilizing the correspondent enzymes to each of the catecholamines as seen in FIG. 7. The first to be done is the use of PNMT to detect the prescence of Norepinephrine in a variety of solutions such as purified in 1M Phosphate Buffer Saline (PBS) and Redox Probe, and different % volumes of blood, while PNMT is immobilized on a gold disk working electrode with a platinum counter and Ag⁺/VAgCl reference electrode is a set up similar to the one shown in FIG. 8 shown with a glassy carbon working electrode.

For a subcutaneous sensor, such as the one suggested in this disclosure, to be commercially viable and successful, the sensor must embody some critical characteristics. Those characteristics include: has a quick response time, is multiplexable (can detect multiple markers simultaneously), has a low limit of detection (highly sensitive), is highly specific (does not sense similar molecules in addition to the desired target), is low in cost, and is user-friendly. All of these characteristics together in one product should be a sustainable product, especially if this device is adaptable to sense a multitude of biomarkers. Adaptability would be easy if the needles were designed to be interchangeable for another needle with different proteins; by this mechanism, theoretically any protein can be used to detect any marker in the body Also, this interchangeability would be beneficial for continuous use in the hospital case for prolonged uses or to monitor out-patient levels for some time after the patient has left the hospital.

The applications for this continuous subcutaneous sensor are mainly in the hospital and military settings. If a patient is known to have sustained Traumatic Brain Injury, then the catecholamines in addition to other biomarkers, such as the interleukins to monitor for inflammation, can be monitored for information regarding the progress and state of the injury. If this sensor were then interfaced with an automatic drug delivery system, inflammation can be counteracted before the brain can inflame to the point of hitting the skull causing secondary damage and necrosis, while also diminishing the neuroplasticity of the brain. If this can be achieved, hospital stays would be shorter and more positive outcomes viable. Also, glucose and lactate can be monitored to detect aerobic and anaerobic metabolism as other indications of TBI.

Another application would be for soldier monitoring for stress, dehydration, TBI, etc. This sensor could have a wireless component which can alert commanding officers of soldier's physiological states without impeding the soldier's activity. In the event a soldier is injured, medical attention can be swift if it is known what type of injury has occurred. Furthermore, this sensor could be used as a continuous monitor in the out-patient sense. If a patient has recently had a heart attack, the sensor could continuously monitor stress and other biomarkers related to heart dysfunction without being at the hospital (driving down costs and possible exposure to hospital-acquired infections).

Some refinements and activities include integrating and multiplexing, needle fabrication, leeching experiments, and animal testing. Integrating and multiplexing is performed after all activities for each detecting protein has been characterized and EIS has been used on physiological ranges of the catecholamines in purified and blood solutions. Needle fabrication and general set up of the needles sensor requires some attention as far as what gauge, length, type, and which configuration of needle is best for this application. Tests to determine these characteristics include testing in engineered tissue (polymer and hydrogel molds) with flowing blood, and purified testing to ensure specificity and sensitivity are maintained.

Other experiments include leeching tests to ensure no reagents enter the blood stream in vivo. After in vitro biocompatibility testing has been concluded, animal testing may begin. Additional considerations for the whole sensor include interfacing wireless components and possibly drug delivery devices.

With reference to the disclosure herein, a method is described to continuously measure electrochemical activity of one or more biochemical or molecular markers. The method includes the steps of attaching a substrate having electronics for measuring electrochemical activity and a plurality of electrodes operably connected to the electronics such that the electrodes are in contact with the subcutaneous layer of a subject's skin; and measuring a biochemical process associated with the one or more biochemical or molecular markers in vivo by detecting an electrochemical signal in the subcutaneous skin layer using the plurality of electrodes.

Preferably, the electrochemical signal is generated such that multiple frequencies are multiplexed together on a carrier wave and sent down a counter electrode while recording and demultiplexing the signal from a working electrode. Also preferably, the substrate is flexible and adhesive, such as the “bandage” embodiment depicted herein.

The one or more markers to be measured include Dopamine, Epinephrine, Norepinephrine, Glucose, Lactate, Cortisol and other indicators of stress. Moreover, multiplexed electrochemical impedance-time signals can be used to interrogate an electrochemical cell formed by the electrodes.

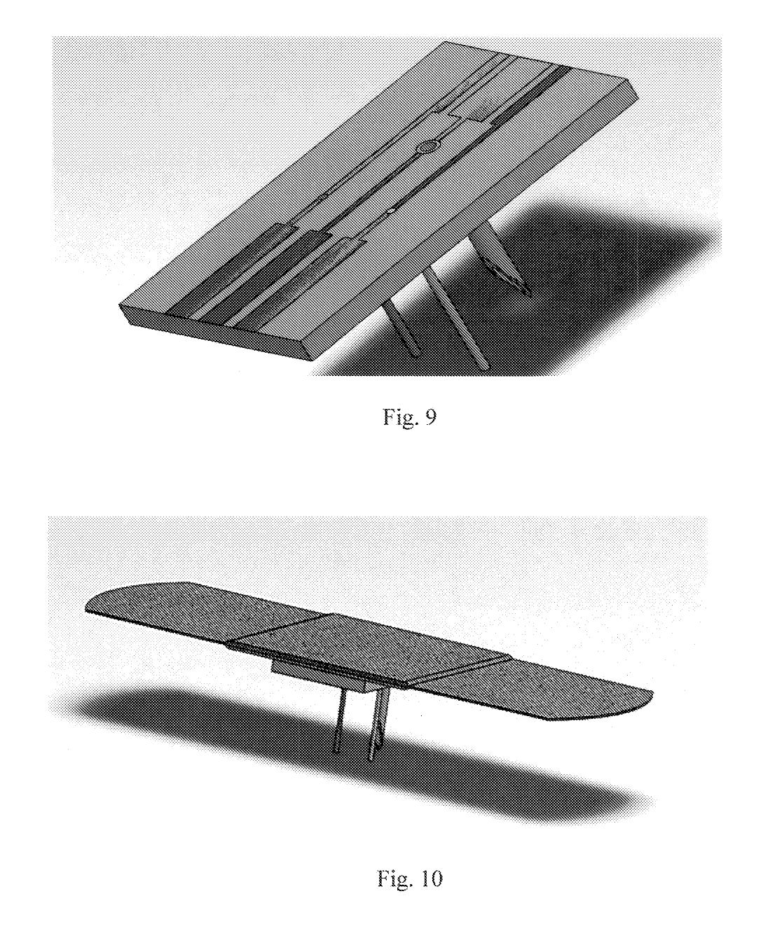

A device also is described to continuously measure electrochemical activity of one or more biochemical or molecular markers. The device can, for example, take the following structure. The device includes a substrate having electronics adapted for measurement of electrochemical activity and a plurality of electrodes, with the electrodes being attached to the substrate, operably connected to the electronics, and adapted to penetrate the skin to a subcutaneous layer. The electrodes may be comprised of electroactive polymers, plastics, metals, ceramics and the like. For example, devices can be embodied as shown in FIGS. 9 and 10.

The substrate for the device ideally has an adhesive layer that sticks to the epidermis, a hard printed circuit board layer that contains the mechanical and electrical connections for the sensors, and instrumentation layer of sensing electrochemical electronics are enclosed and sealed to prevent damage to the components inside.

The sensor electronics are multiple signal generators, a multiplexer to mix the signals, conditioning circuitry, potentiostat to record the impedance signals, a demultiplexer, A/D converters, storage memory, on board memory, microcontroller and processor, as well as battery power. These electronics can be standardized parts (all currently available from public sources), surface mount technologies, or flexible electronics.

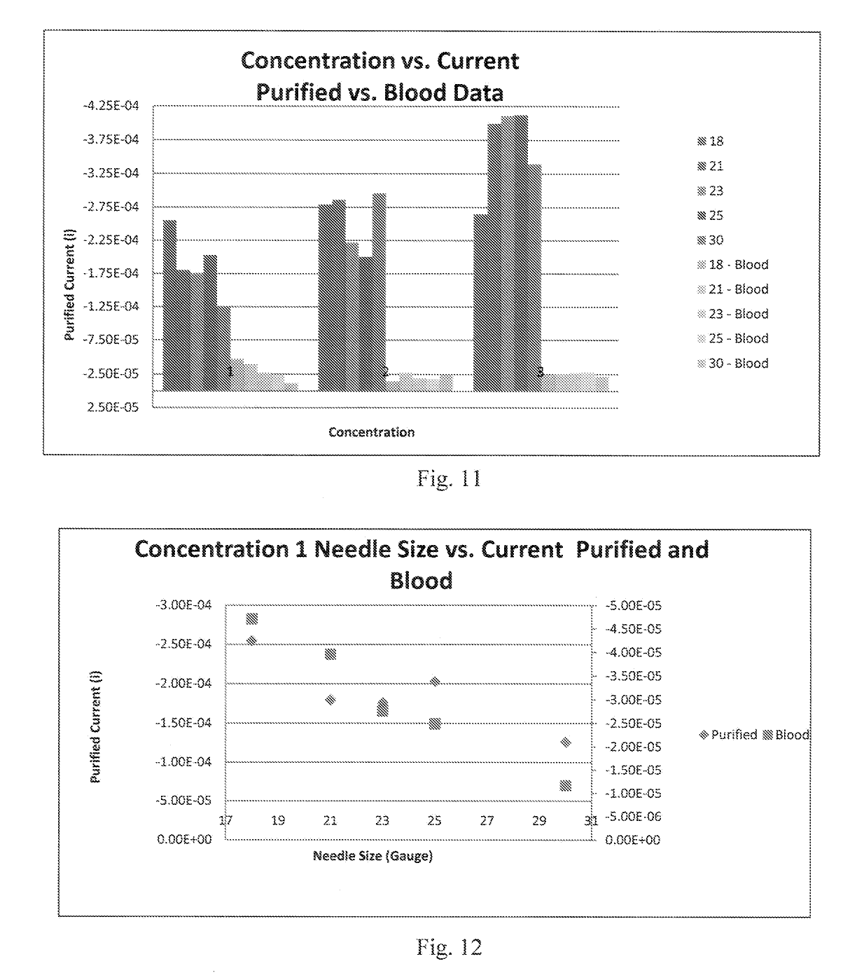

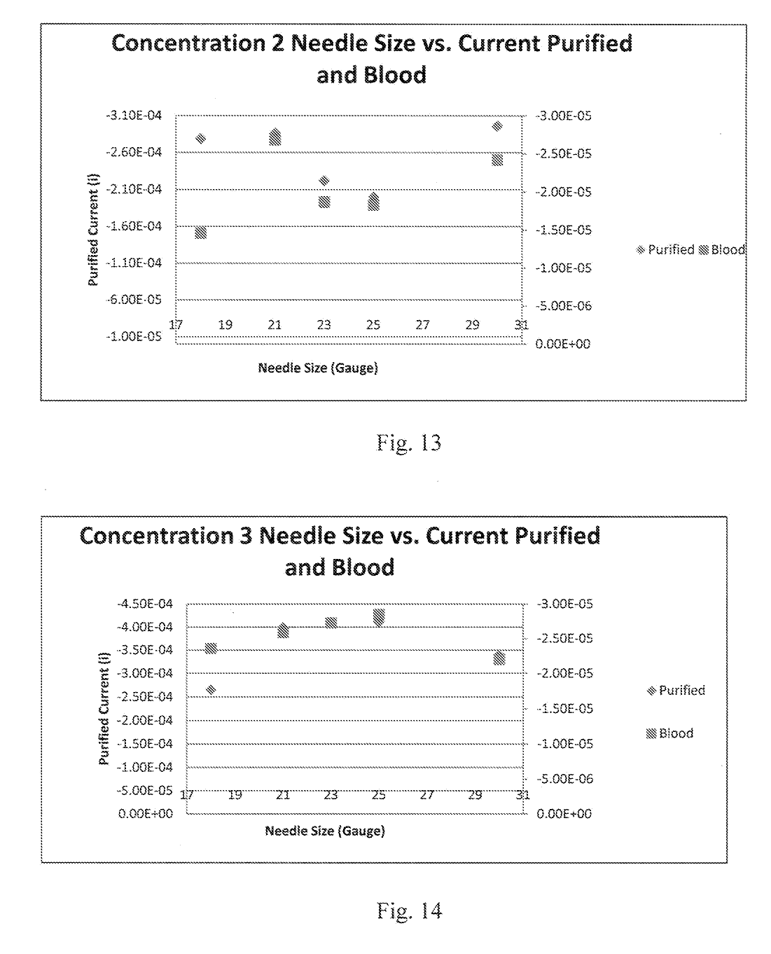

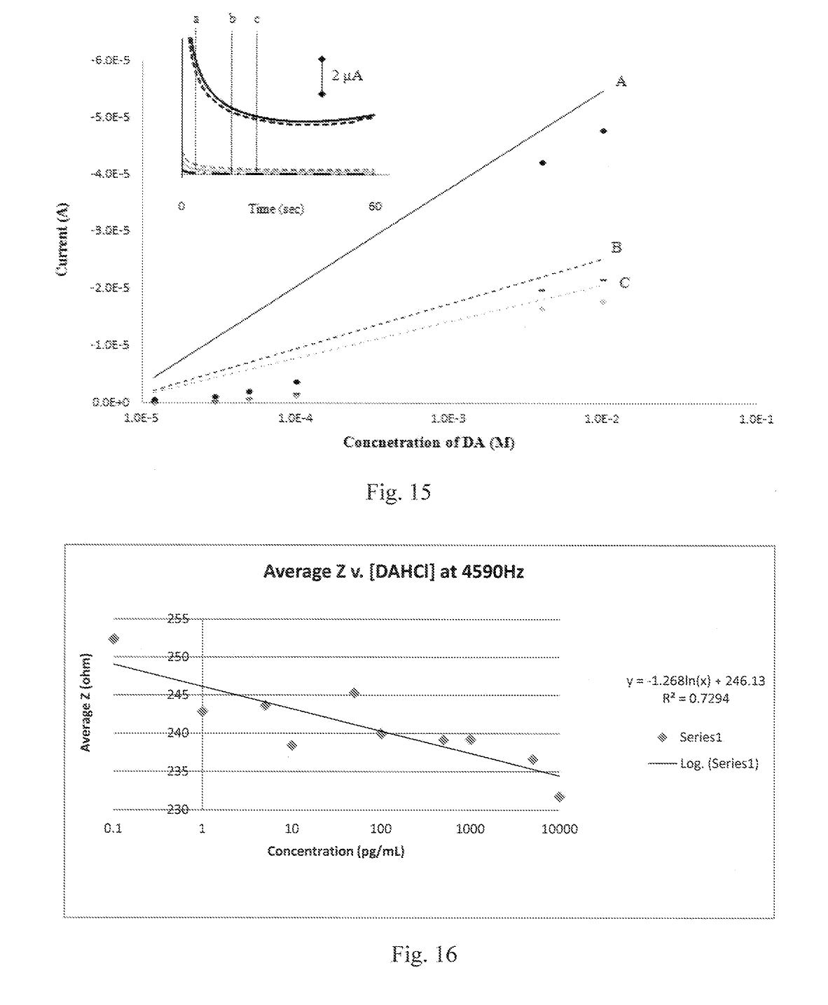

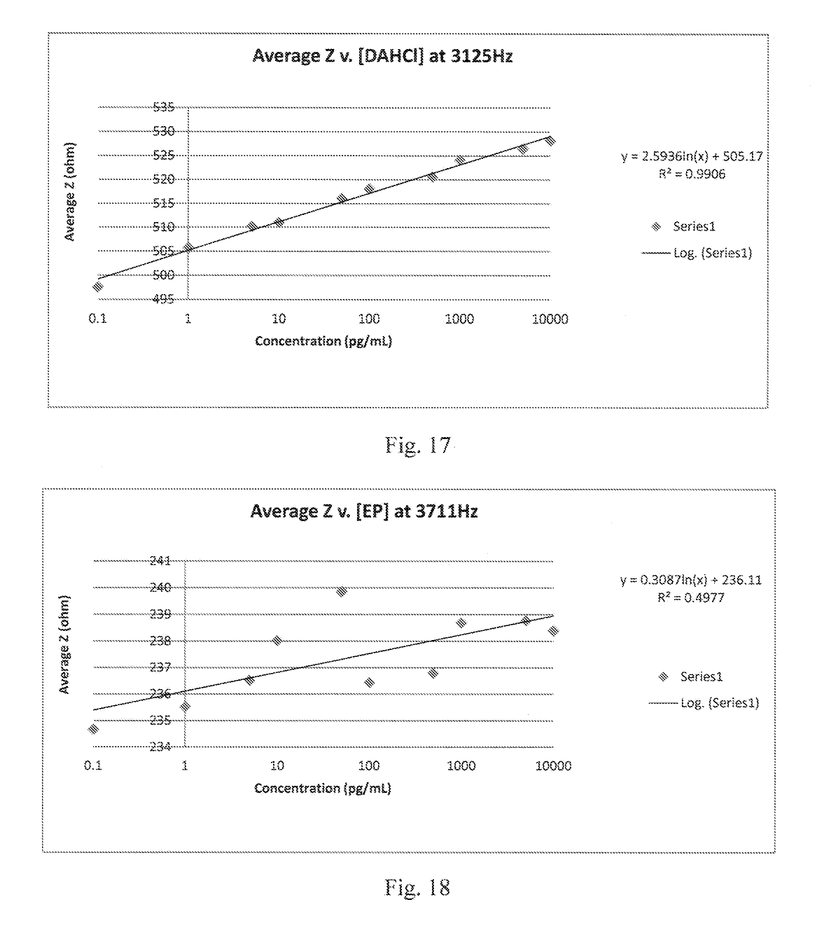

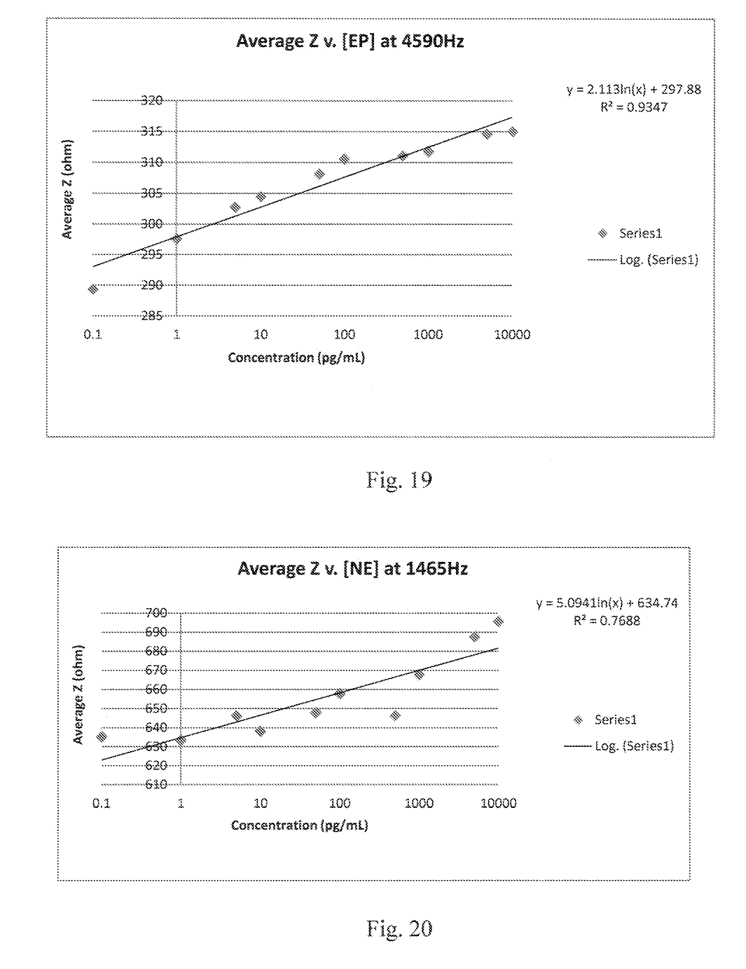

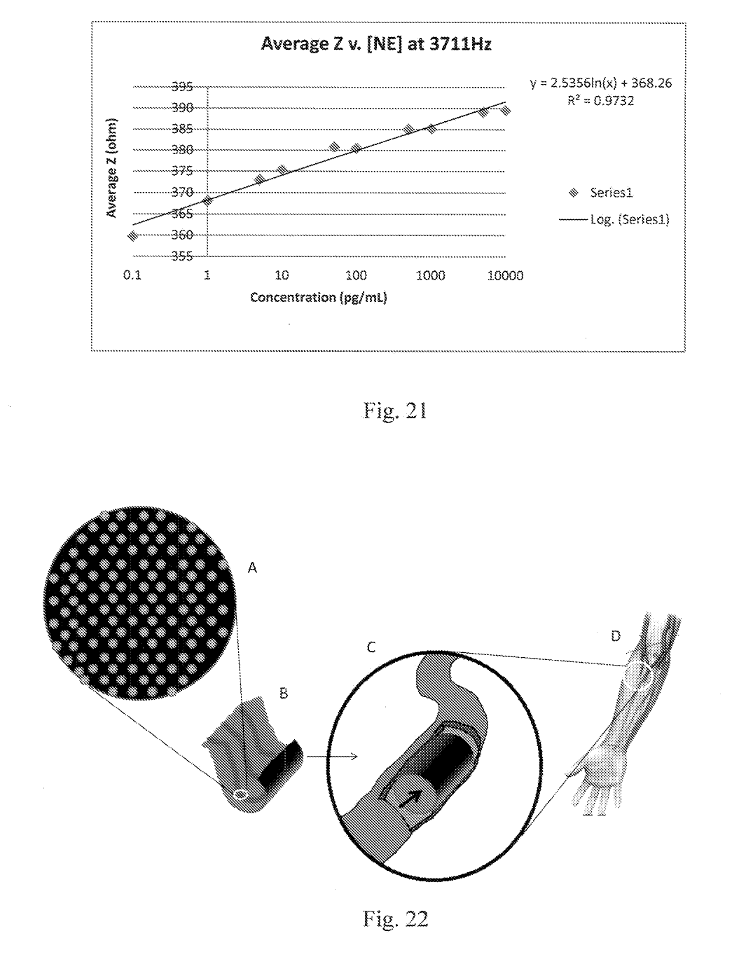

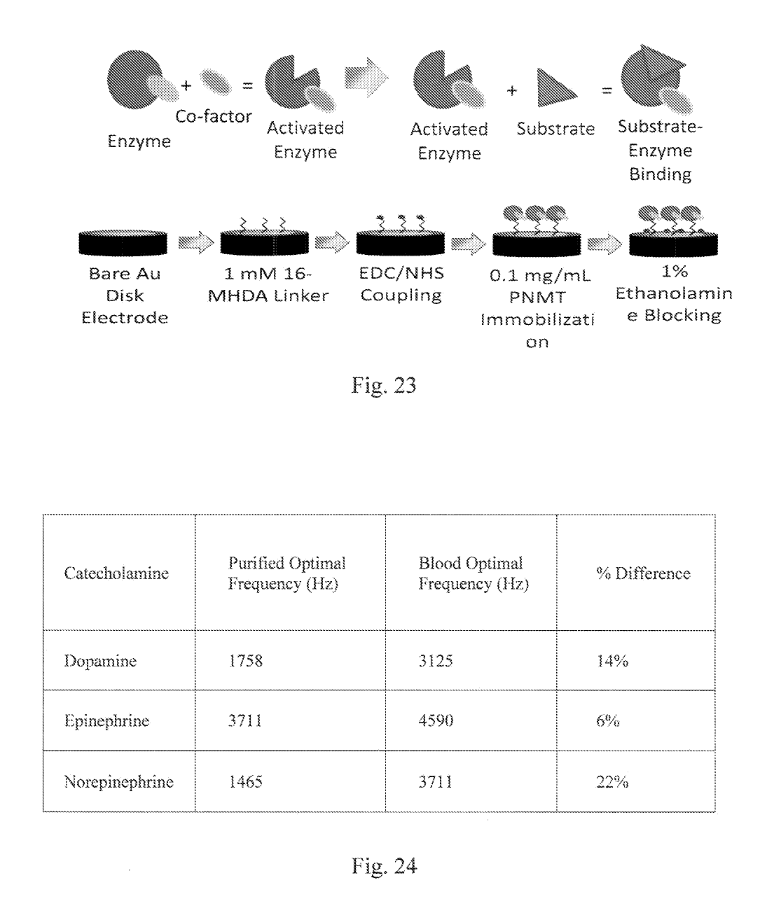

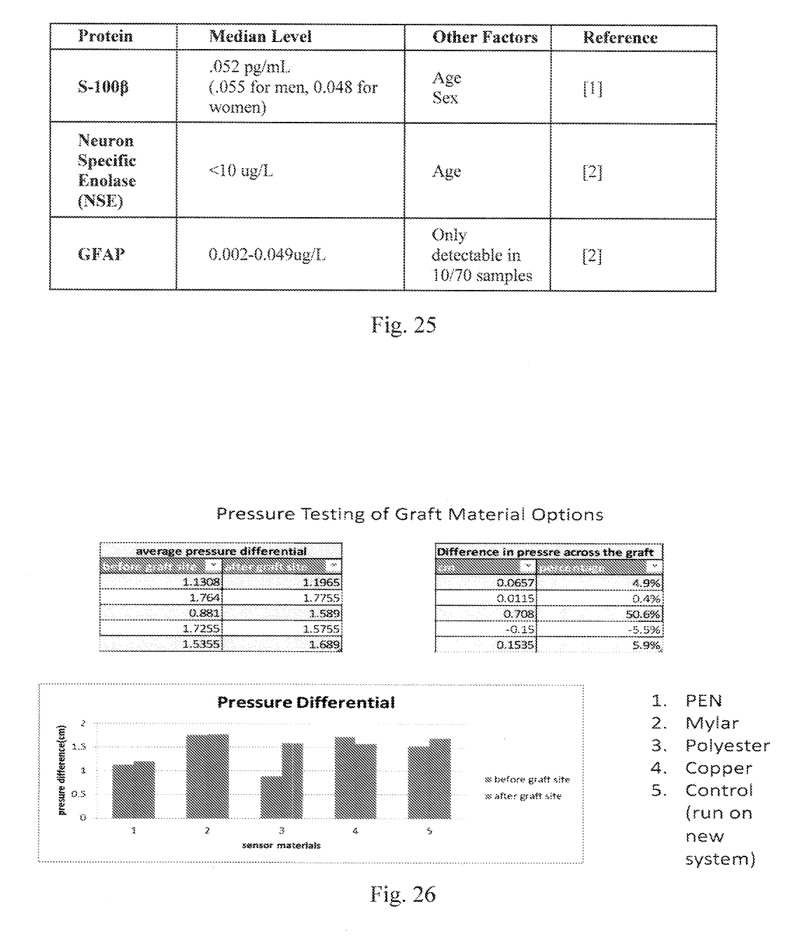

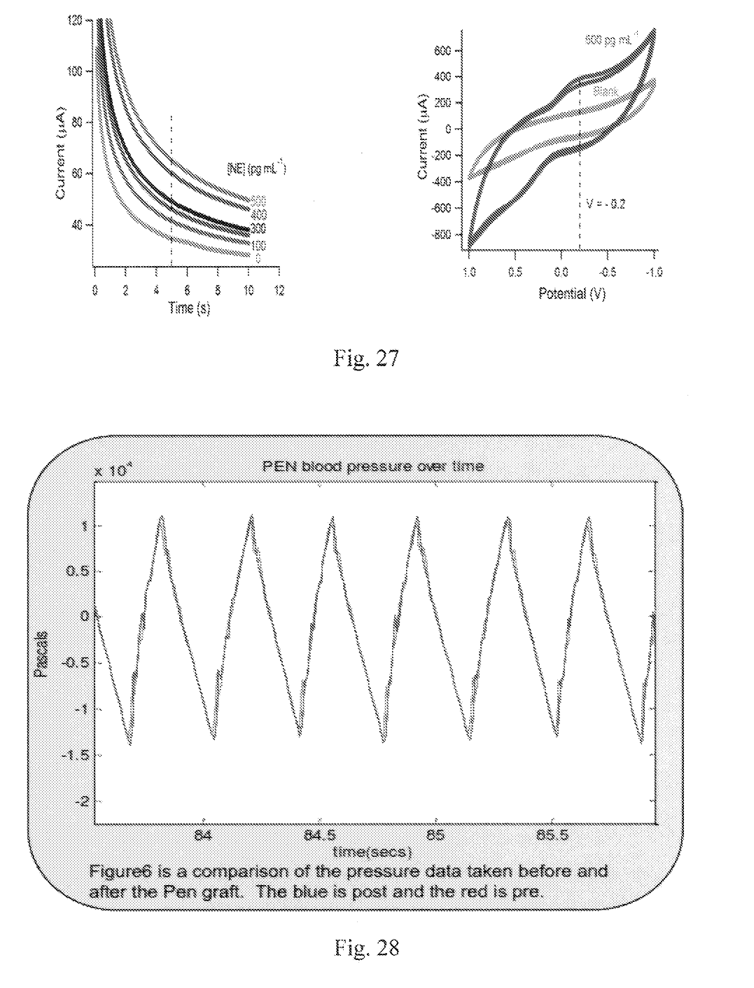

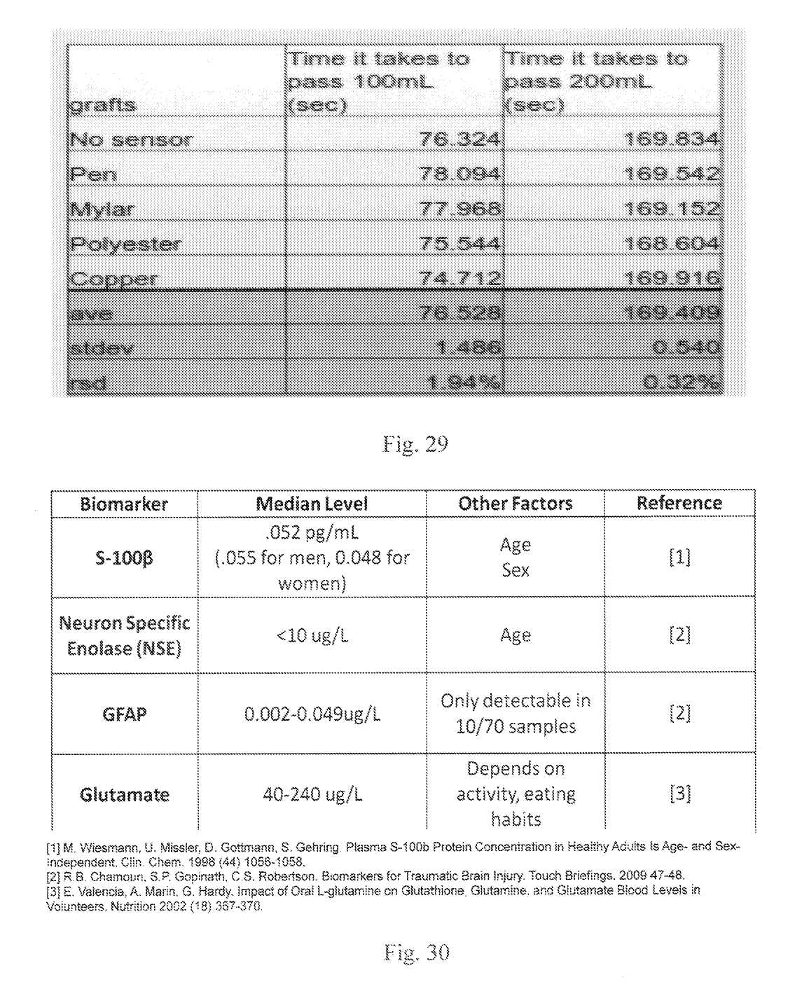

FIGS. 11-30 relate to device testing and embodiments encompassing intravenous and other implantable sensor designs.

### REFERENCES

[1] “CDC—Statistics—Traumatic Brain Injury—Injury Center.” Centers for Disease Control and Prevention. 5 May 2011. Web. 4 Nov. 2011. <http://www.cdc.gov/traumaticbraininjury/statistics.html>.
[2] Ng, T.; Arias, A. C.; Daniel, J. H.; Garner, S.; Lavery, L.; Sambandan, S.; Whiting, G. L. Flexible printed sensor tape for diagnostics of mild traumatic brain injury. IDTechEx Printed Electronics Asia; 2009 Sep. 30-Oct. 1; Tokyo, Japan.
[3] Droppert, Pieter. “Detecting Brain Injury Using a Sensor That Changes Color|Biotech Strategy Blog.” Biotech Strategy Blog—Pharma Marketing Strategy, New Product Development, Medical Science & Innovation. 26 Apr. 2011. Web. 17 Oct. 2011. <http://biotechstrategyblog.com/2011/04/how-nanotechnology-may-revolutionize-the-detection-of-traumatic-brain-injury-using-a-sensor-that-changes-color.html/>.
[4] Sauser, Brittany. “Fighting Head Trauma in Iraq—Technology Review.” Technology Review: The Authority on the Future of Technology. MIT, 18 Sep. 2007. Web. 17 Oct. 2011. <http://www.technologyreview.com/infotech/19396/>.
[5] Wang, Dr. Kevin, Dr. Weihong Tan, Dr. Firas Kobeissy, and Dr. Stephen Lamer. “Traumatic Brain Injury Nanosensor.” Center for Nano-Bio Sensors. UF-CNBS 205 Particle Science & Technology, 10 Oct. 2007. Web. 4 Nov. 2011. <http://cnbs.centers.uffedu/research/brain.asp>.
[6] “User Guides:Paradigm® Revel™ User Guide.” Medtronic Download Library. Medtronic, 10 Feb. 2010. Web. 1 Nov. 2011. <www.medtronicdiabetes.net/support/download-library/user-guides>.
[7] “Traumatic Brain Injury (TBI) Task Force Report.” Army Medicine. US Army Medical Department, 17 Jan. 2008. Web. 17 Oct. 2011. <http://www.armymedicine.army.mil/prr/tbitfr.html>.

All embodiments of any aspect of the invention can be combined with other embodiments of any aspect of the invention unless the context clearly dictates otherwise.

Various changes in the details and components that have been described may be made by those skilled in the art within the principles and scope of the invention herein described in the specification and defined in the appended claims. Therefore, while the present invention has been shown and described herein in what is believed to be the most practical and preferred embodiments, it is recognized that departures can be made therefrom within the scope of the invention, which is not to be limited to the details disclosed herein but is to be accorded the full scope of the claims so as to embrace any and all equivalent processes and products.

1. A method to continuously measure electrochemical activity of one or more biochemical or molecular markers, comprising the steps of:
attaching a substrate having electronics for measuring electrochemical activity and a plurality of electrodes such that said electrodes are in contact with the subcutaneous layer of a subject's skin;
and measuring a biochemical process associated with said one or more biochemical or molecular markers in vivo by detecting an electrochemical signal in said subcutaneous layer using said plurality of electrodes.

2. The method of claim 1, wherein an electrochemical signal is generated such that multiple frequencies are multiplexed together on a carrier wave and sent down a counter electrode while recording and demultiplexing the signal from a working electrode.

3. The method of claim 1, wherein the substrate is flexible and adhesive.

4. The method of claim 1, wherein said one or more markers include Dopamine, Epinephrine, Norepinephrine, Glucose, Lactate, Cortisol.

5. The method of claim 1, wherein multiplexed electrochemical impedance-time signals are used to interrogate an electrochemical cell formed by said electrodes.

6. A device adapted to measure electrochemical activity of a biochemical or molecular marker, comprising: a substrate having electronics adapted for measurement of electrochemical activity and a plurality of electrodes, said electrodes being attached to said substrate, operably connected to said electronics, and adapted to penetrate the skin to a subcutaneous layer.

7. A device of claim 6, wherein the electrodes are comprised of electroactive polymers and plastics, metals, ceramics.
